(19) United States
(12) Patent Application Publication
(10) Pub. No.: US 2009/0183602 A1
(43) Pub. Date: Jul. 23, 2009

(54) TATTOO MACHINE

(76) Inventor: Derek S. CROCKETT, Charlotte, NC (US)

Correspondence Address: Clements Bernard PLLC, 1901 Roxborough Road, Suite 300, Charlotte, NC, 28211, US

(21) Appl. No.: 12/357,576

(22) Filed: Jan. 22, 2009

Related U.S. Application Data
(60) Provisional application No. 61/022,592, filed on Jan. 22, 2008.

(51) Int. Cl. B43K 5/00; A61B 17/34

(52) U.S. Cl. 81/9.22; 606/186

(57) ABSTRACT

The present disclosure provides a tattoo machine including a frame, an armature bar coupled to a needle bar, at least one electromagnetic coil, and a dampening device disposed to the frame. The dampening device absorbs vibrational forces exerted on the frame of the tattoo machine and thereby helps minimize wear and tear as well as increases the longevity of the machine. The absorption of vibrational forces increases the comfort of the tattoo artist along with their accuracy.

### CROSS-REFERENCE TO RELATED APPLICATION(S)

This application claims the benefit of priority of U.S. Provisional Application No. 61/022,592 filed Jan. 22, 2008, and entitled “TATTOO MACHINE”, the contents of which are incorporated in full by reference herein.

### FIELD OF THE INVENTION

The present invention relates generally to a tattoo machine. In particular, the present invention relates to a tattoo machine having a bushing to absorb the vibrational forces exerted on the tattoo machine during operation for preventing premature failure and increasing user comfort and “feel.”

### BACKGROUND OF THE INVENTION

Tattoos are created by injecting ink into the skin. A tattoo artist employs a tattoo machine for creating a tattoo. Conventionally, the tattoo machine includes alternating electromagnetic coils that are used to move a reciprocating needle that injects ink into the skin. The needle punctures the skin between 50 and 3,000 times per minute, and penetrates the skin by approximately a millimeter. The needle injects ink into the penetration hole, creating a permanent marking of the skin.

The electromagnetic coil assembly is the most important component of any tattoo machine that employs electromagnetism to reciprocate the needle assembly. Conventional tattoo machines employ electromagnetic coils with a cylindrical steel core wrapped in copper wire (typically American wire gage (“awg”) 24) through which AC current is passed to create an electromagnet. The alternating polarity of the electromagnet alternately attracts and repels an armature bar at a high rate of reciprocation. A tattoo needle assembly is attached to the reciprocating armature bar. The tattoo needle is then used to intra-dermally inject ink into a human or animal subject. Prior art coils use non-removable retaining washers and o-rings to retain the wires.

The large number of punctures per minute, results in the reciprocating armature bar of the tattoo machine having to create one up/down cycle each time the needle punctures the skin. That is a relatively large number of cycles per minute, and an even greater number of cycles per hour when the needle punctures the skin between 50-3000 times per minute. These cycles place a lot of stress on the components of the tattoo machine. As a result, there is a likelihood that these components will fail, resulting in the tattoo machine failing prematurely. In addition, it is imperative that a tattoo machine have the right “feel” and be comfortable for the tattoo artist to use, both while creating a single tattoo and over the span of months and years.

The wear and tear on previous tattoo machines is evident from their short lifespan. Traditionally these machines experience such strong vibrational forces that the machine wears out, breaks, or needs replacement parts within months. The strong vibrational forces also create discomfort for the tattoo artist. The strong vibrational forces can be detrimental to the tattoo artist's accuracy as well.

Thus, there exists a need for an improved tattoo machine design which dampens the vibrational forces exerted through use of the tattoo machine.

### BRIEF SUMMARY OF THE INVENTION

In various exemplary embodiments, the present invention provides a tattoo machine having a dampening device to absorb the vibrational forces exerted on the tattoo machine for preventing premature failure and increasing user comfort and “feel.” The present invention seeks to dampen the vibrational forces exerted on the machine, by employing bushings at the intersections of the electromagnetic coils and the frame, thereby minimizing the wear and tear. Additionally, the dampening of the vibrational forces also increases the comfort and accuracy of the tattoo artist. The tattoo machine of the present invention includes a frame, an armature bar attached to the frame, a needle bar coupled to the armature bar, at least one electromagnetic coil connected to the frame, and a dampening device, such as a bushing, attached at the intersection of the electromagnetic coil and the frame or to any other bore located on the frame adapted to receive a threaded bolt or similar bolt like structure.

In an exemplary embodiment of the present invention, a tattoo machine includes a frame; an armature bar attached to the frame; at least one electromagnetic coil connected to the frame and disposed adjacent to the armature bar; and a dampening device disposed to the frame operable to absorb vibrational forces from the at least one electromagnetic coil. The tattoo machine can further include a needle bar coupled to the armature bar; and an armature spring that is engaged to the frame. Optionally, the at least one electromagnetic coil includes two electromagnetic coils including a first electromagnetic coil and a second electromagnetic coil. Alternatively, the dampening device includes a first bushing and a second bushing, wherein the first bushing is attached at an intersection of the first electromagnetic coil and the frame and the second bushing is attached at an intersection of the second electromagnetic coil and the frame. The dampening device can include a material coated on the frame operable to absorb vibrational forces from the at least one electromagnetic. The tattoo machine can further include at least one needle engaged to the needle bar for puncturing the skin and depositing ink in the puncture hole. Optionally, the tattoo machine further includes a rabbit ear with a screw engaged therein, wherein a portion of the rabbit ear is bent approximately 90°. The dampening device can be attached at an intersection of the electromagnetic coil and the frame.

In another exemplary embodiment of the present invention, a tattoo machine includes a frame; an armature bar attached to the frame; a needle bar coupled to the armature bar; a first electromagnetic coil connected to the frame; a second electromagnetic coil connected to the frame; an armature spring engaged with the frame; a first bushing attached at an intersection of the first electromagnetic coil and the frame; and a second bushing attached at an intersection of the second electromagnetic coil and the frame. The tattoo machine further includes an armature spring that is engaged to the frame. Alternatively, the tattoo machine further includes a clip cord. The tattoo machine can further include at least one needle engaged to the needle bar for puncturing the skin and depositing ink in the puncture hole. The tattoo machine of further includes a rabbit ear with a screw engaged therein, wherein a portion of the rabbit ear is bent approximately 90°. Alternatively, the tattoo machine further includes a third bushing at a bore on the frame.

In yet another exemplary embodiment of the present invention, a tattoo machine includes a frame; an armature bar attached to the frame; a needle bar coupled to the armature bar; at least one electromagnetic coil connected to the frame; and a bushing employed at a bore located on the frame adapted to receive a threaded bolt or similar bolt like structure. The tattoo machine can further include a clip cord. Optionally, the tattoo machine further includes a rabbit ear with a screw engaged therein, wherein a portion of the rabbit ear is bent approximately 90°. The at least one electromagnetic coil can include two electromagnetic coils with a first electromagnetic coil and a second electromagnetic coil, and the tattoo machine can further include a second bushing, wherein the second bushing is attached at an intersection of the second electromagnetic coil and the frame. The tattoo machine can further include at least one needle engaged to the needle bar for puncturing the skin and depositing ink in the puncture hole; and an armature spring that is engaged with the frame.

### BRIEF DESCRIPTION OF THE DRAWINGS

The present invention is illustrated and described herein with reference to the various drawings, in which like reference numbers denote like system components, respectively, and in which:

FIG. 1 is a perspective view of a tattoo machine, according to an exemplary embodiment of the present invention; and

FIG. 2 is another perspective view of the tattoo machine of FIG. 1, according to an exemplary embodiment of the present invention.

### DETAILED DESCRIPTION OF THE INVENTION

In various exemplary embodiments, the present invention provides a tattoo machine having a dampening device to absorb the vibrational forces exerted on the tattoo machine for preventing premature failure and increasing user comfort and “feel.” The present invention seeks to dampen the vibrational forces exerted on the machine, by employing bushings at the intersections of the electromagnetic coils and the frame, thereby minimizing the wear and tear. Additionally, the dampening of the vibrational forces also increases the comfort and accuracy of the tattoo artist.

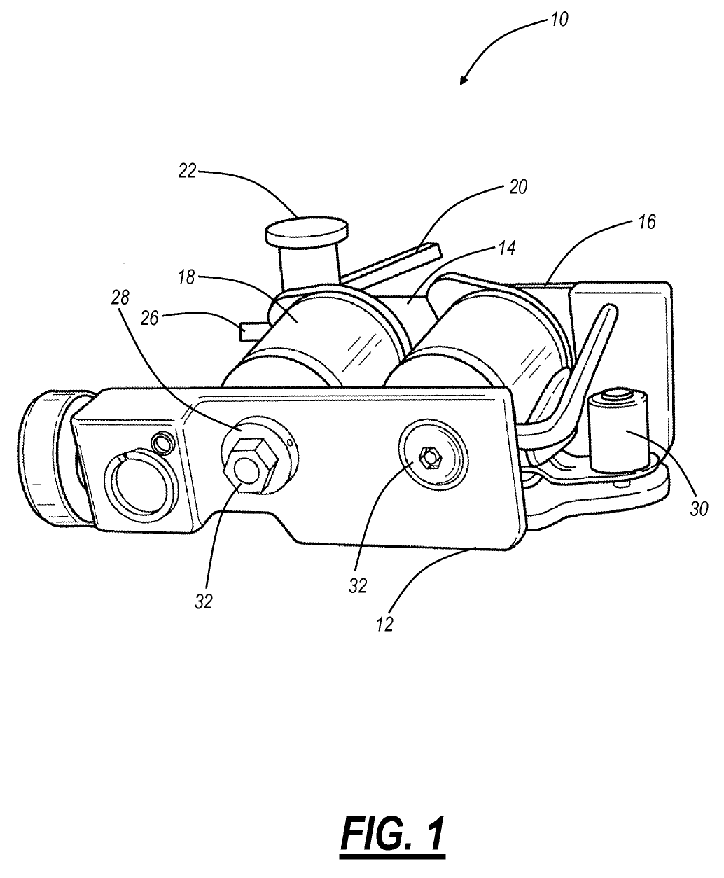

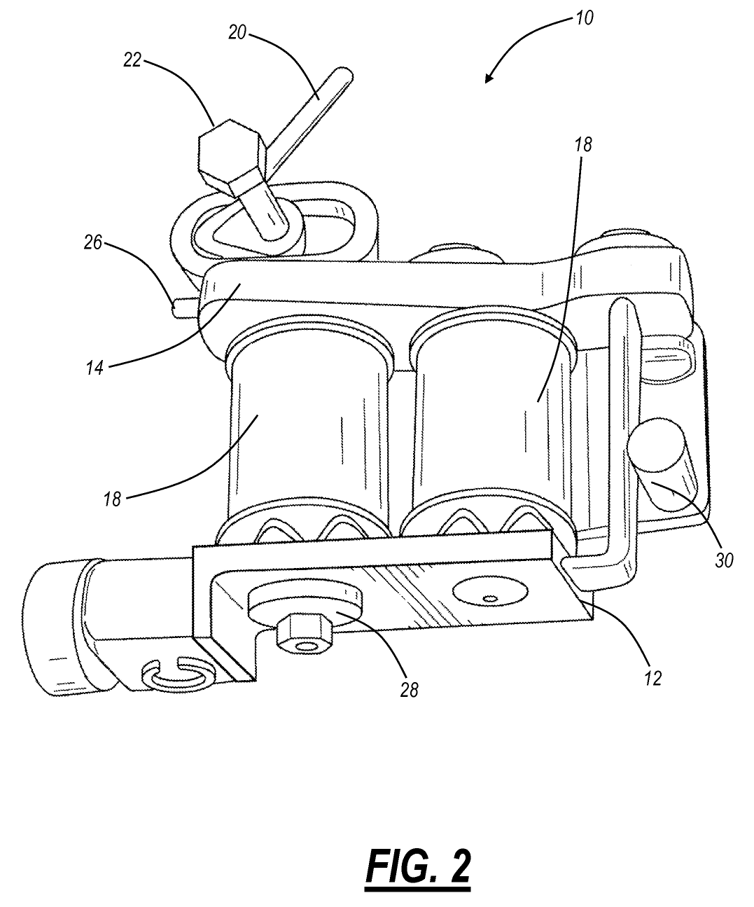

Referring to FIGS. 1 and 2, an exemplary tattoo machine 10 is illustrated according to an exemplary embodiment of the present invention. The tattoo machine 10 generally includes a frame 12, an armature bar 14, an armature spring 16, and electromagnetic coils 18. The armature bar 14 is coupled to a needle bar (not shown). Generally, a clip cord (not shown) supplies power to the electromagnetic coils 18, supplying power to the tattoo machine 10. The clip cord can be designed to be interchangeable between various tattoo machines.

An electromagnetic coil (or simply a “coil”) is formed when a conductor (usually a solid copper wire) is wound around a core or form to create an inductor or electromagnet. One loop of wire is usually referred to as a turn, and a coil consists of one or more turns. For use in an electronic circuit, electrical connection terminals called taps are often connected to a coil. Coils are often coated with varnish and/or wrapped with insulating tape to provide additional insulation and secure them in place. A completed coil assembly with taps etc. is often called a winding. A transformer is an electromagnetic device that has a primary winding and a secondary winding that transfers energy from one electrical circuit to another by magnetic coupling without moving parts. The term tickler coil usually refers to a third coil placed in relation to a primary coil and secondary coil. A coil tap is a wiring feature found on some electrical transformers, inductors and coil pickups, all of which are sets of wire coils. The coil tap(s) are points in a wire coil where a conductive patch has been exposed (usually on a loop of wire that extends out of the main coil body). As self induction is larger for larger coil diameter the current in a thick wire tries to flow on the inside. The ideal use of copper is achieved by foils. Sometimes this means that a spiral is a better alternative. Multilayer coils have the problem of interlayer capacitance, so when multiple layers are needed the shape needs to be radically changed to a short coil with many layers so that the voltage between consecutive layers is smaller (making them more spiral like).

The frame 12 can be composed of a conductive metal. Such suitable metals include copper, iron, steel, brass, or the like. Accordingly, the frame 12 of the tattoo machine 10 can conduct electricity. Alternatively, the frame 12 can be made of a nonconductive material, and the frame 12 can include a yoke or similar device that connects the electromagnetic coils 18 to a power supply to allow conduction of electricity from the power source.

Positioned on top of the base is a rabbit ear 20 with a screw 22 positioned on the rabbit ear 20. For example, the rabbit ear 20 is angled at about 90° to hold the electromagnetic coils 18 in place. The armature spring 16 is attached to the top portion of the frame 12, and the armature bar 14 is connected to the armature spring 16. For example, the portion of the screw 22 on the rabbit ear 20 is insulated from the frame 12 for protecting the tattoo artist.

The tattoo machine 10 can be powered in two ways. First, electricity can be conducted through the screw 22 positioned on the frame 12. Second, electricity can be conducted through the frame 12 by the armature spring 16. The frame 12 includes a contact spring 24 that is positioned above one end of the electromagnetic coils 18 and is in an engaged arrangement with the armature spring 16. When a power supply, such as a clip cord, is connected to the tattoo machine 10, the current flows through the screw 22 and contact spring 24, thus completing a circuit. Additionally, the present invention contemplates additional powering mechanisms for the tattoo machine 10 as are known in the art.

The tattoo machine 10 can include a set of contact points. One contact point is located on the armature spring 16, and the other contact point is located on the screw 22. The contact points allow current to flow through the electromagnetic coils 18 to the armature bar 14. The contact points function as a type of switch that is controlled by the contact spring 24 to turn the tattoo machine 10 on and off readily.

A capacitor 30 regulates the current flow. The purpose of the capacitor 30 is to prevent the electromagnetic coils 18 from wearing out prematurely. The capacitor 30 charges when the points are open. In other words, when the electromagnetic coils 18 are not in contact with the points, allowing the capacitor 30 to charge. The difference in voltage across the capacitor 30 when the points are open is negligible.

A needle bar (not shown) may be inserted into a first hole 26 located in front of the rabbit ear 20. A needle may be engaged to the needle bar by soldering or the like. Alternatively, a plurality of needles may be engaged to the needle bar in various configurations, depending upon the pattern the tattoo artist desires. A sanitary tube (not shown) is engaged to the needle bar for storing ink before it is transferred to the needle and ultimately the skin of an individual. The sanitary tube sucks up ink in a capillary fashion, before the needles are infused with the ink. The needle or needles are drawn into the sanitary tube by the reciprocating motion of the needle bar, allowing the needle to load up on ink. The needle penetrates the skin, wherein the ink is deposited, and then the ink returns to the sanitary tube to receive more ink to be deposited into the skin of an individual.

One end of the electromagnetic coils 18 is connected to the frame 12 such as through a screw 32 or the like, and the other end is positioned adjacent the armature bar 14. Current flows through the electromagnetic coils 18 from the power supply. Current flowing through the electromagnetic coils 18 causes the electromagnetic coils 18 to become electromagnetic. When the electromagnetic coils 18 become electromagnetic the electromagnetic coils 18 pull down the armature bar 14 through an electromagnetic force thereby enabling the armature bar 14 to reciprocate based on the turning on and off of the electromagnetic coils 18. As one can imagine, the force exerted on the armature bar 14 by the electromagnetic coils 18 occurs a high number of times during the course of the application of a tattoo. Therefore, the tattoo machine 10 has a tendency to fail prematurely as discussed herein and vibrate accordingly.

To prevent premature failure and reduce vibrational forces, the present invention employs the use of at least one bushing 28 engaged to the point of contact between the electromagnetic coils 18 and the frame 12. The bushing 28 is a cylindrical lining designed to constrict and restrain motion of mechanical parts thereby reducing mechanical vibrations. The bushing 28 acts as a dampening device between the electromagnetic coils 18 and the frame 12. The bushing 28 can be composed of any compressible material or it can be a damper. Exemplary materials for the bushing 28 can include PTFE (Teflon), graphite, graphite/metal (Graphalloy), ceramic, steel, bronze, rubber, polyurethane, and the like. The bushing can be shaped like a washer but is not usually made entirely of metal.

In FIGS. 1 and 2, the at least one bushing 28 is illustrated as a washer-shaped device disposed between the screw 30 and the frame 12. The present invention contemplates the bushing 28 on one or on all of the electromagnetic coils 18. Additionally, the bushing 28 could be on any bore adapted to receive a threaded bolt or the like for dampening the forces exerted thereon between the frame 12 and the electromagnetic coils 18.

For example, one type of bushing is a threaded insert, a hardened metal insert with a threaded fixing hole which allows one assembly to be fixed to another by means of a screw or threaded bolt. The use of threaded insert avoids the need for a separate nut and washer on the other side of the fixed material. Such inserts are usually fitted into sheet material by a tool which operates on a similar principle to riveting; alternatively, some rivets can themselves incorporate a bushing.

The bushing 28 dampens the force exerted by the electromagnetic coils 18 on the reciprocating armature bar 14. For example, the bushing 28 acts as a shock absorber from the vibrational forces exerted by the electromagnetic coils 18 to the frame. The bushing 28 can be employed at only one contact between the electromagnetic coils 18 and frame 12. Alternatively, a bushing can be employed at all contacts between the electromagnetic coils 18 and the frame 12. In yet another exemplary embodiment, a bushing may be employed at any bore adapted to receive a threaded bolt or the like for dampening the forces exerted thereon. Alternatively, if the tattoo machine 10 includes three or more electromagnetic coils 18, a bushing could be employed at all of the contacts between the electromagnetic coils 18 and the frame 12.

The present invention also contemplates other mechanisms acting as a dampening device between the electromagnetic coils 18 and the frame 12. For example, the frame 12 could be coated with rubber, a plastic material, or the like operable to absorb vibrations between the electromagnetic coils 18 and the frame 12. Alternatively, a spring element could be disposed to the electromagnetic coils 18 to act as a shock absorber.

During operation, current flows between the screw 22 and the contact spring 24, and through the frame 14, the armature spring 16, and contact spring 24, thus completing the circuit. The completion of the circuit causes the electromagnetic coils 18 to exert a force upon the armature bar 14, forcing the armature bar 14 downward. As the armature bar 14 moves downward, the needle bar moves downwards also, resulting in the needle on the end of the needle bar to move downward. If a tattoo artist is performing his craft upon an individual, this downward movement allows the needle to puncture the skin and deposit a drop of ink within the puncture hole. While the armature bar 14 is in the downward position, the circuit is broken, resulting in the armature bar 14 returning to its original position. When the armature bar 14 returns to its original position, the circuit is completed, resulting in the electromagnetic coils 18 exerting a force upon the armature bar 14 and forcing the armature bar 14 downward.

The clip cord can be a spring coiled U-cable that connects the electromagnetic coils 18 to a DC power supply. An acceptable DC power supply is 6-12 V. The clip cord is designed to be interchangeable between various tattoo machines. This enables the tattoo artist to disconnect and connect various tattoo machines adapted for a predetermined purpose from just one clip cord. One end of the clip cord is inserted into a standard electrical outlet, while the other end is engaged to the tattoo machine 10. Preferably, the end of the clip cord that is engaged to the tattoo machine 10 has a lip or notch that retains the clip cord firmly in place within a cavity of the tattoo machine 10.

A foot pedal (not shown) is connected to the clip cord. The food pedal allows the tattoo artist to use the foot pedal to commence operation of the tattoo machine 10. The foot pedal may also be used to control the speed of the tattoo machine 10, meaning that the foot pedal may be used to increase or decrease the number of times the needle punctures the skin of an individual. The importance of the foot pedal is to allow the tattoo artist to operate the tattoo machine 10 without the use of his hands, since the hands are used to hold the tattoo machine 10 and stretch the skin during application of the ink.

It will be appreciated by those skilled in the art that the type and weight of the armature bar 14, the gauge and shape of the contact spring 24, the type of armature spring 16 can vary depending on the preferences and requirements of the tattoo artist. In addition, the return force of the armature spring 16 and armature bar 14 will cause various levels of flex that could result in the on/off cycle that is off balance. The on/off cycle is more properly defined as the amount of time the circuit is open and the amount of time the circuit is closed. Preferably, the off balance should generally range around 45% to 50%, meaning the circuit is open about the same duration as the circuit is closed. However, a slight off balance may be preferred by the tattoo artist depending upon personal preference while performing his craft.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A tattoo machine, comprising:
a frame;
an armature bar attached to the frame;
at least one electromagnetic coil connected to the frame and disposed adjacent to the armature bar; and
a dampening device disposed to the frame operable to absorb vibrational forces from the at least one electromagnetic coil.

2. The tattoo machine of claim 1, further comprising:
a needle bar coupled to the armature bar; and
an armature spring that is engaged to the frame.

3. The tattoo machine of claim 1, wherein the at least one electromagnetic coil comprises two electromagnetic coils comprising a first electromagnetic coil and a second electromagnetic coil.

4. The tattoo machine of claim 3, wherein the dampening device comprises a first bushing and a second bushing, wherein the first bushing is attached at an intersection of the first electromagnetic coil and the frame and the second bushing is attached at an intersection of the second electromagnetic coil and the frame.

5. The tattoo machine of claim 1, wherein the dampening device comprises a material coated on the frame operable to absorb vibrational forces from the at least one electromagnetic.

6. The tattoo machine of claim 1, further comprising at least one needle engaged to the needle bar for puncturing the skin and depositing ink in the puncture hole.

7. The tattoo machine of claim 2, further comprising a rabbit ear with a screw engaged therein, wherein a portion of the rabbit ear is bent approximately 90°.

8. The tattoo machine of claim 1, wherein the dampening device is attached at an intersection of the electromagnetic coil and the frame.

9. A tattoo machine, comprising:
a frame;
an armature bar attached to the frame;
a needle bar coupled to the armature bar;
a first electromagnetic coil connected to the frame;
a second electromagnetic coil connected to the frame;
an armature spring engaged with the frame;
a first bushing attached at an intersection of the first electromagnetic coil and the frame; and
a second bushing attached at an intersection of the second electromagnetic coil and the frame.

10. The tattoo machine of claim 9, further comprising an armature spring that is engaged to the frame.

11. The tattoo machine of claim 9, further comprising a clip cord.

12. The tattoo machine of claim 9, further comprising at least one needle engaged to the needle bar for puncturing the skin and depositing ink in the puncture hole.

13. The tattoo machine of claim 9, further comprising a rabbit ear with a screw engaged therein, wherein a portion of the rabbit ear is bent approximately 90°.

14. The tattoo machine of claim 9, further comprising a third bushing at a bore on the frame.

15. A tattoo machine, comprising:
a frame;
an armature bar attached to the frame;
a needle bar coupled to the armature bar;
at least one electromagnetic coil connected to the frame; and
a bushing employed at a bore located on the frame adapted to receive a threaded bolt or similar bolt like structure.

16. The tattoo machine of claim 15, further comprising a clip cord.

17. The tattoo machine of claim 15, further comprising a rabbit ear with a screw engaged therein, wherein a portion of the rabbit ear is bent approximately 90°.

18. The tattoo machine of claim 15, wherein the at least one electromagnetic coil comprises two electromagnetic coils comprising a first electromagnetic coil and a second electromagnetic coil.

19. The tattoo machine of claim 18, further comprising a second bushing, wherein the second bushing is attached at an intersection of the second electromagnetic coil and the frame.

20. The tattoo machine of claim 15, further comprising:
at least one needle engaged to the needle bar for puncturing the skin and depositing ink in the puncture hole; and
an armature spring that is engaged with the frame.
